(19) United States
(12) Patent Application Publication
(10) Pub. No.: US 2025/0388535 A1
(43) Pub. Date: Dec. 25, 2025

(54) SULFONATED AND SULFATED ARYL-ALKYL COMPOUNDS USEFUL AS SURFACTANTS

(71) Applicant: SPECIALTY OPERATIONS FRANCE, Lyon (FR)

(72) Inventor: Olivier Back, Saint-Fons (FR)

(73) Assignee: SPECIALTY OPERATIONS FRANCE, Lyon (FR)

(21) Appl. No.: 18/879,712

(22) PCT Filed: Jun. 20, 2023
(86) PCT No.: PCT/EP2023/066638

§ 371 Date: Dec. 27, 2024

(30) Foreign Application Priority Data
Jul. 1, 2022 (EP) 22182457.6

(51) Int. Cl. C07C 309/24; C07C 305/06; C11D 1/22

(52) U.S. Cl. CPC C07C 309/24; C07C 305/06; C11D 1/22

(57) ABSTRACT

The present invention relates to sulfonated and sulfated compounds obtainable from aryl aliphatic ketones, a process to produce such compounds and the use of these compounds as surfactants, alone or in admixture with other surfactants.

This application claims priority to the European application filed on Jul. 1, 2022 with the number 22182457.6, the whole content of which being incorporated herein by reference for all purposes.

### TECHNICAL FIELD AND BACKGROUND

The present invention relates to sulfonated and sulfated compounds obtainable from aryl aliphatic ketones, processes to produce such compounds, and the use of such compounds as surfactants, alone or in admixture with other surfactants.

WO 2018/229285 A1 discloses a process for the cross-ketonization between an aryl carboxylic acid and an aliphatic carboxylic acid using a metal-based compound. The resulting aryl aliphatic ketones can be used for the preparation of surfactants.

It is generally desirable to provide new families of surfactants with good toxicity and environmental profile that further display a good biodegradable profile.

### SUMMARY OF THE PRESENT INVENTION

One object of the present invention is thus to provide compounds useful as surfactants that have good toxicity and environmental profiles, as well as good, preferably improved biodegradability profiles.

This object is solved by a compound of formula I as defined in claims 1 to 8 and by a composition comprising a mixture of two or more compounds of formula I as defined in claims 9 and 10 and by a composition comprising at least one compound of formula I and at least one further surfactant, wherein the at least one further surfactant is not a compound of formula I, as defined in claim 11.

It is a further object of the present invention to provide a process for the preparation of a compound of formula I as defined in any one of claims 1 to 8.

This object is solved by a process as defined in any one of claims 12 to 16 of the present application.

Finally, it is an object of the present invention to provide a specific use of the compounds of formula I according to any one of claims 1 to 8 or the compositions according to any one of claim 9 to 11.

This object is solved by the subject matter claimed in claim 17 of the present application.

### DETAILED DESCRIPTION

### The Compounds of the Present Invention

The present invention provides compounds of formula I

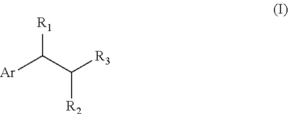

wherein Ar represents an aryl group;
R₁ is H or a group of formula OR wherein R is H or SO₃X wherein X is an alkali metal, alkali-earth metal or ammonium of general formula R′R″R″′R″″N wherein R′, R″, R″′ and R″″ are independently chosen from hydrogen or a hydrocarbyl group which can be optionally substituted and/or interrupted by one or more heteroatom containing groups;
R₂ is H or SO₃X wherein X is an alkali metal, alkali-earth metal or ammonium of general formula R′R″R″′R″″N wherein R′, R″, R″′ and R″ ″ are independently chosen from hydrogen or a hydrocarbyl group which can be optionally substituted and/or interrupted by one or more heteroatom containing groups;
with the provisio that
R₁ and R₂ are different groups
when R₂ is SO₃X, R₁ is H or OR with R is H
when R₂ is H, R₁ is OR with R is SO₃X
R₃ is a C₂-C₂₆ aliphatic group.

The compounds of the present invention have been found to provide a good toxicity and environmental profile. Furthermore, the compounds of the present invention display a good biodegradability profile, which has been found to be improved compared to the corresponding aryl-alkyl ketone sulfonates Ar—C(═O)—CH(SO₃X)—R₃ of the general formula VI. Aryl-alkyl ketone sulfonates are e.g. known from WO 2018/229285 A1.

An “aryl group” (Ar) in the context of the present invention means an aromatic group, i.e. a cyclic conjugated unsaturated hydrocarbon ring having a number of delocalized π electrons following the Huckel rule; it also encompasses polycyclic aromatic groups, wherein the cyclic aromatic rings can be fused or linked by a C—C linkage; it also encompasses heteroaromatic groups, i.e. cyclic or polycyclic conjugated unsaturated rings containing one or more heteroatoms having a number of delocalized π electrons following the Huckel Rule (for example, furane, pyridine, pyrrole, as will be described in further detail below). The aryl group can optionally be further substituted by one or more functional groups.

An example for an aromatic ring as aryl group Ar is phenyl, which can optionally be further substituted by one or more functional groups. Ar may represent a substituted phenyl group of the following formula

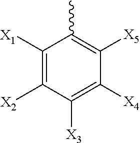

wherein X₁, X₂, X₃, X₄ and X₅ which can be the same or different represent
   hydrogen or a C₁-C₂₄ linear or branched hydrocarbon group having 1 to 24 carbon atoms which can be optionally substituted and/or interrupted by one or more heteroatoms or heteroatom containing groups,
   halogen,
   hydroxy (—OH) or alkoxy group (—OR) wherein R is a linear or branched hydrocarbon group having 1 to 24 carbon atoms which can be optionally substituted and/or interrupted by one or more heteroatoms or heteroatom containing groups,
   amino group (—NRR′) wherein R and R′ independently represent hydrogen or a linear or branched hydrocarbon group having 1 to 24 carbon atoms which can be optionally substituted and/or interrupted by one or more heteroatoms or heteroatom containing groups,
   acyl group (—(C═O)—R) wherein R represents hydrogen or a C₁-C₂₄ linear or branched hydrocarbon group having 1 to 24 carbon atoms which can be optionally substituted and/or interrupted by one or more heteroatoms or heteroatom containing groups,
   carboxyl (—COOH) or alkoxycarbonyl group (—(C═O)—OR) wherein R represents a C₁-C₂₄ linear or branched hydrocarbon group having 1 to 24 carbon atoms which can be optionally substituted and/or interrupted by one or more heteroatoms or heteroatom containing groups,
   carbamoyl group (—(C═O)—NRR′) wherein R and R′ independently represent hydrogen or a linear or branched hydrocarbon group having 1 to 24 carbon atoms which can be optionally substituted and/or interrupted by one or more heteroatoms or heteroatom containing groups,
   alkylsulfonyl group (—SO₂—R) or alkylsulfinyl group (—SO—R) or alkylthio group (—S—R) wherein R represents a C₁-C₂₄ linear or branched hydrocarbon group having 1 to 24 carbon atoms which can be optionally substituted and/or interrupted by one or more heteroatoms or heteroatom containing groups.

Examples for heteroaromatic rings as Aryl group Ar include pyridyl, furanyl, pyrrolyl, thiophenyl, pyrazolyl, imidazolyl, benzimidazolyl, indolyl, quinolinyl, isoquinolinyl, purinyl, pyrimidinyl, thiazolyl, pyrazinyl, pyridazinyl, oxazolyl and triazolyl, which may be further substituted by one or more functional groups.

For example, Ar may represent an optionally substituted 2-pyridyl, 3-pyridyl or 4-pyridyl group of formula

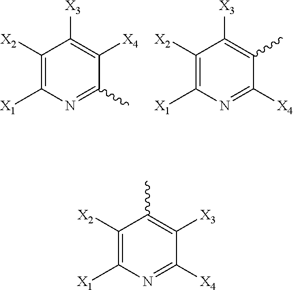

wherein X₁, X₂, X₃ and X₄ which can be the same or different have the same meaning as above described.

Ar may also represent an optionally substituted furan-2-yl or furan-3-yl group of formula

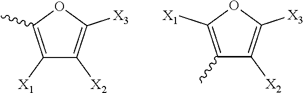

wherein X₁, X₂ and X₃ which can be the same or different have the same meaning as above described.

Ar may also represent an optionally substituted 1H-pyrrol-2-yl or 1H-pyrrol-3-yl group of formula:

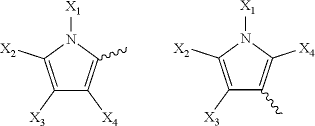

wherein X₁, X₂, X₃ and X₄ which can be the same or different have the same meaning as above described.

Ar may also represent an optionally substituted thiophen-2-yl or thiophen-3-yl group of formula:

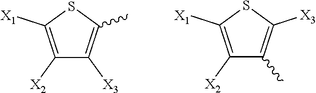

In some embodiments, substituents Xᵢ and Xᵢ₊₁ beared by 2 adjacent carbons of the phenyl, the pyridyl, the furanyl, the pyrrolyl or the thiophenyl, form together an optionally substituted cyclic moiety said cyclic moiety being an aromatic, hetero-aromatic or non-aromatic group.

In a preferred embodiment of the present invention, the aryl group Ar is a phenyl group.

“R₃” in the context of the present invention generally represents a C₂-C₂₆ aliphatic group, preferably a C₂-C₁₈ aliphatic group, more preferably a C₆-C₁₆ aliphatic group, even more preferably a C₁₀-C₁₆ aliphatic group.

The aliphatic group R₃ may be free of any double bond and of any triple bond. Alternatively, the aliphatic group R₃ may also comprise at least one C—C double bond and/or at least one C—C triple bond.

The aliphatic group R₃ may be linear or branched, and is preferably a linear alkyl group.

The aliphatic group R₃ is advantageously chosen from alkyl groups, alkenyl groups, alkanedienyl groups, alkanetrienyl groups. Preferably, it is chosen from alkyl, alkenyl and alkanetrienyl groups.

More preferably, the aliphatic group R₃ is chosen from alkyl and alkenyl groups, generally from C₂-C₂₆ alkyl and C₂-C₂₆ alkenyl group, preferably from C₂-C₁₈ alkyl and C₂-C₁₈ alkenyl groups, more preferably from C₆-C₁₆ alkyl and C₆-C₁₆ alkenyl groups, more preferably from C₁₀-C₁₆ alkyl and C₁₀-C₁₆ alkenyl groups.

Even more preferably, R₃ represents an alkyl group, generally a C₂-C₂₆ alkyl group, preferably a C₂-C₁₈ alkyl group, more preferably a C₆-C₁₆ alkyl group, which can be branched or linear alkyl groups, and are preferably linear alkyl groups.

The cation X in the SO₃X group of the compounds of the present invention is selected from alkali metals, alkali-earth metals and ammonium.

Alkali metals suitable as group X for use in the present invention include lithium (Li), sodium (Na), potassium (K), rubidium (Rb) and cesium (Cs), with lithium, sodium and potassium being preferred alkali metals, and sodium being a particularly preferred alkali metal.

Alkali-earth metals suitable as group X for use in the present invention include beryllium (Be), magnesium (Mg), calcium (Ca), strontium (Sr) and barium (Ba), with magnesium and calcium being preferred alkali-earth metals.

Ammonium suitable as group X for use in the present invention have the general formula R′R″R″′R″″N, wherein R′, R″, R″′ and R″″ are independently chosen from hydrogen or a hydrocarbyl group which can be optionally substituted and/or interrupted by one or more heteroatom containing groups. The term “hydrocarbyl” as used herein comprises aryl and aliphatic groups, which can be optionally substituted and/or interrupted by one or more heteroatom containing groups. Such heteroatom containing groups comprise nitrogen containing groups, such as primary, secondary, and tertiary amines, as well as oxygen containing groups, such as hydroxyl groups and ether groups. Preferred ammonium suitable as group X for use in the present invention include e.g. NH₄, N(CH₃)₄, triethanolammonium.

Preferred compounds of formula I are defined as follows:
R₁ is OSO₃X wherein X is defined as above and in the claims, R₂ is H, R₃ is a linear alkyl group C₆-C₁₆; and
R₁ is OH, R₂ is SO₃X wherein X is defined as above and in the claims, R₃ is a linear alkyl group C₆-C₁₆; and
R₁ is H, R₂ is SO₃X wherein X is defined as above and in the claims, R₃ is a linear alkyl group C₆-C₁₆.

More preferred compounds of formula I are defined as follows:
Ar is phenyl, R₁ is OSO₃X wherein X is sodium, R₂ is H, R₃ is a linear alkyl group C₆-C₁₆; and
Ar is phenyl, R₁ is OH, R₂ is SO₃X wherein X is sodium, R₃ is a linear alkyl group C₆-C₁₆; and
Ar is phenyl, R₁ is H; R₂ is SO₃X, wherein X is sodium, R₃ is a linear alkyl group C₆-C₁₆.

The biodegradability of the compounds of the present invention can be determined following a well-known standard protocol according to the OECD guidelines, such as for example the OECD 301F protocol. An alternative protocol developed by the inventors for the determination of the biodegradability of the inventive compounds disclosed herein is described in the examples section.

### The Compositions of the Present Invention

The present invention also concerns compositions comprising a mixture of two or more compounds of formula I as defined herein. Such compositions can e.g. be prepared either by mixing the respective compounds together or they are obtainable from the process of the present invention described herein below.

In one embodiment, the composition of the present invention for example comprises a mixture of a first compound of formula I wherein R₁ is OH, R₂ is SO₃X wherein X is defined as above and in the claims, R₃ is a linear alkyl group C₆-C₁₆, and a second compound of formula I wherein R₁ is H, R₂ is SO₃X wherein X is defined as above and in the claims, R₃ is a linear alkyl group C₆-C₁₆. Preferably, said first compound is defined as follows: Ar is phenyl, R₁ is OH, R₂ is SO₃X wherein X is sodium, R₃ is a linear alkyl group C₆-C₁₆; and said second compound is defined as follows: Ar is phenyl, R₁ is H, R₂ is SO₃X wherein X is sodium, R₃ is a linear alkyl group C₆-C₁₆. Such compositions are obtainable from alternative b. of the process of the present invention as described herein below.

The present invention furthermore concerns compositions that comprise at least one compound of formula I together with a further surfactant, wherein said surfactant is not a surfactant in accordance with the compound of formula I. Such compositions can be prepared by mixing the components of the composition together.

### The Process of the Present Invention

The present invention furthermore provides a process for the preparation of a compound of formula I as described herein.

### Overview

The process of the present invention starts from a mixture of Ar—COOH (II) and R₃—CH₂—COOH (III) to produce an aryl aliphatic ketone of formula IV: Ar—C(═O)—CH₂—R₃ (IV), wherein Ar and R₃ are defined as above and in the claims. The process of the present invention then further comprises the derivatization of the aryl aliphatic ketone of formula IV: Ar—C(═O)—CH₂—R₃ (IV) by means of a hydrogenation-sulfatation sequence (alternative a.) or a sulfonation-hydrogenation sequence (alternative b.) to produce the compound of formula I.

For the preparation of a compound of formula I wherein R₁ is OSO₃X wherein X is as defined above and in the claims, R₂ is H, R₃ is a C₂-C₂₆ aliphatic group, the process according to the present invention is carried out according to alternative a. which involves the following steps:
a. decarboxylative cross-ketonization between Ar—COOH (II) and R₃—CH₂—COOH (III), wherein Ar and R₃ are as defined in any one of the preceding claims, in presence of a metal based catalyst thus obtaining an aryl aliphatic ketone of formula IV: Ar—C(═O)—CH₂—R₃ (IV), wherein Ar and R₃ are as defined above,
b. hydrogenation of the aryl aliphatic ketone of formula IV obtained at step a. in presence of H₂ and a catalyst thus obtaining a secondary alcohol of formula V: Ar—CH(OH)—CH₂—R₃ (V), wherein Ar and R₃ are as defined above,
c. sulfatation of the secondary alcohol of formula V obtained at step b. with a sulfating agent followed by a neutralization with XOH or X(OH)₂.

For the preparation of a first compound of formula I wherein R₁ is OH, R₂ is SO₃X wherein X is defined as above and in the claims, and R₃ is a C₂-C₂₆ aliphatic group;
or a second compound of formula I wherein R₁ is H, R₂ is SO₃X wherein X is defined as above and in the claims, and R₃ is an aliphatic C₂-C₂₆ group;
or a mixture of the first compound and second compound of formula I, the process of the present invention is carried out according to alternative b. which involves the following steps:
a. decarboxylative cross-ketonization between Ar—COOH (II) and R₃—CH₂—COOH (III), wherein Ar and R₃ are as defined in any one of the preceding claims, in presence of a metal based catalyst thus obtaining an aryl aliphatic ketone of formula IV: Ar—C(═O)—CH₂—R₃ (IV), wherein Ar and R₃ are as defined above,
b. sulfonation of the aryl aliphatic ketone of formula IV obtained at step a. with a sulfonating agent followed by a neutralization with XOH or X(OH)₂ thus obtaining an aryl aliphatic ketone sulfonate of formula VI: Ar—C(═O)—CH(SO₃X)—R₃ (VI) wherein Ar, X and R₃ are as defined above,
c. hydrogenation of the aryl aliphatic ketone sulfonate of formula VI obtained at step b. in presence of H₂ and a catalyst.

### Step a.—Decarboxylative Cross-Ketonization

A decarboxylative cross-ketonization reaction according to step a. of the process of the present invention between aryl carboxylic acid Ar—COOH (II) and aliphatic carboxylic acid R₃—CH₂—COOH (III) acid to provide aryl aliphatic ketones of formula IV: Ar—C(═O)—CH₂—R₃ (IV), wherein Ar and R₃ are defined as above and in the claims, forms part of both alternatives a. and b. of the process of the present invention.

The starting materials Ar—COOH (II) and R₃—CH₂—COOH (III) used in the process of the present invention are either commercially available or can be synthesized by the skilled person in view of his/her common general knowledge.

The decarboxylative cross-ketonization is preferably carried out as catalytic decarboxylative cross-ketonization. Such a procedure is known from WO 2018/229285 A1 (see e.g. page 4, lines 9-34 of WO 2018/229285 A1). The catalytic decarboxylative cross-ketonization of aryl- and aliphatic carboxylic acids as described in detail in WO 2018/229285 A1 can be analogously applied in the context of the present invention in order to provide aryl aliphatic ketones of formula IV: Ar—C(═O)—CH₂—R₃ (IV).

The skilled person in view of his/her common general knowledge is also able to modify a decarboxylative cross-ketonization procedure applied in the art for the purpose of the present invention, if necessary.

Alternative a.: Steps b. And c.—Hydrogenation-Sulfatation-Neutralization

In the following, the hydrogenation-sulfatation sequence according to steps b. and c. of alternative a. of the process of the present will be further described.

Step b. of alternative a.: In the process of the present invention according to alternative a., the aryl aliphatic ketone of formula IV: Ar—C(═O)—CH₂—R₃ (IV) is hydrogenated in the presence of H₂ and a catalyst, thereby obtaining a secondary alcohol of formula V: Ar—CH(OH)—CH₂—R₃ (V), wherein Ar and R₃ are as defined above and in the claims.

The catalytic hydrogenation of aryl aliphatic ketones in order to obtain the corresponding aryl aliphatic alcohol is a standard reaction commonly known to the skilled person.

In the process of the present invention, the hydrogenation step is carried out in the presence of a metal based catalyst, preferably a transition metal based catalyst, which is typically palladium on charcoal (Pd/C).

In the present invention, the hydrogenation step can be carried out in a reactor/autoclave with hydrogen gas (H₂)-pressures up to 100 bar (such as e.g. 80 bar, 60 bar, 40 bar or 20 bar) at temperatures up to 150° C., preferably up to 100° C., such as e.g. 90° C.

The catalytic hydrogenation can be carried out in solution using an additional solvent. In that case, the skilled person in view of his/her common general knowledge is able to choose a suitable solvent from the range of standard solvents widely used in organic synthesis. As examples of typical solvents used in catalytic hydrogen reaction one can mention for example: methanol, ethanol, isopropanol, tert-butanol, THF, Me-THF, saturated hydrocarbons, or their mixtures.

The reaction can also be carried out without using a solvent. In that case, the catalytic hydrogenation is typically carried out at temperature where the substrate is present in melted form.

The skilled person in view of his/her common general knowledge is able to control the progress of the reaction using standard techniques, such as NMR.

The skilled person in view of his/her common general knowledge is able to purify the product mixture resulting from the hydrogenation step using common purification and separation techniques, such as distillation methods, precipitation/crystallization methods (if applicable), and chromatographic methods (including e.g. flash chromatography and HPLC).

A procedure for the catalytic hydrogenation of aryl aliphatic ketones is e.g. described in US 2020/0339748 A1 (cf. paragraphs [0265]-[0270] of US 2020/0339748 A1) which can be analogously applied in the process of the present invention. In this preferred protocol, the reaction is conducted without any solvent and a sub-stoichiometric amount of an alkali (e.g. NaOH) is used as an additive in order to enhance the hydrogenation selectivity toward the desired benzylic alcohol derivative.

The skilled person in view of his/her common general knowledge is also able to modify an aryl aliphatic ketone hydrogenation procedure applied in the art for the purpose of the present invention, if necessary.

Step c. of alternative a.: The secondary alcohol of formula V: Ar—CH(OH)—CH₂—R₃ (V) wherein Ar and R₃ are as defined above and in the claims obtained from step b. is then submitted to a sulfatation step with a sulfating agent, said sulfatation being followed by a neutralization with a base XOH or X(OH₂), thereby obtaining the compound of formula I wherein R₁ is OSO₃X wherein X is as defined above and in the claims, R₂ is H, and R₃ is a C₂-C₂₆ aliphatic group.

The sulfatation of secondary alcohols is a standard reaction that forms part of the common general knowledge of the skilled person.

In the context of the present invention, the sulfatation of secondary alcohols of formula V: Ar—CH(OH)—CH₂—R₃ (V) can be carried out using known sulfating agents. Sulfating agents useful in the present invention are e.g. selected from the group consisting of SO₃, chlorosulfonic acid (ClSO₃H), oleum and sulfamic acid (H₃NSO₃). Preferably, SO₃ or chlorosulfonic acid (ClSO₃H) are used as the sulfating agents in the process of the present invention.

The sulfatation can be carried out in solution. The skilled person in view of his/her common general knowledge is able to choose a suitable solvent from the range of standard solvents widely used in organic synthesis, including, but not limited to, e.g. CH₂Cl₂, CHCl₃, CCl₄, pyridine, ethyl acetate, dioxane, diglyme, THF, 2-methyl-THF, hydrocarbons or mixtures thereof.

Alternatively, the sulfatation can be carried out without any solvent when the alcohol is in its melted state.

The sulfatation step can be conducted at temperatures ranging from −20° C. to 120° C., preferably 0° C. to 80° C. Due to the exothermic nature of the reaction, the sulfatation agent is typically added at lower temperatures (e.g. within the range of −20° C. to 10° C.) to the substrate. The temperature is then typically increased in order to accelerate the reaction and increase conversion. In the case when SO₃ is used as a sulfating agent, the sulfatation can be carried out with gaseous SO₃ diluted in dry air or dry nitrogen (for example 1 to 5% v/v) using for example a so called falling film reactor.

The skilled person in view of his/her common general knowledge is able to control the progress of the sulfatation reaction using standard techniques, such as NMR.

An ageing step can optionally be included after the sulfatation step in order to achieve full conversion to the sulfonic acid. Such ageing step can e.g. be conducted at a temperature between 15° C. to 200° C.

The sulfatation step is followed by a neutralization step order to complete salt formation of the desired product, i.e. the compound of formula (I).

The neutralization is carried out using a base compound of the general formula XOH or X(OH)₂, wherein X is an alkali metal, an alkali-earth metal, or an ammonium group as defined above. X can e.g. be Li, Na, K, Cs, NH₄, triethanolammonium, Mg or Ca.

The neutralization using the base compound of the general formula XOH or X(OH)₂ is typically carried out in aqueous solution (or in a solvent mixture to which an aqueous solution of the base compound of the general formula XOH or X(OH)₂ is added). The neutralization is typically carried out at temperatures within the range from 0° C. to 100° C., preferably at temperatures within the range from 0° C. to room temperature. “Room temperature” for the purpose of the present invention is defined herein as ranging from 20° C. to 30° C.

The skilled person in view of his/her common general knowledge is able to purify the product mixture resulting from the sulfatation and subsequent neutralization step using common purification and separation techniques, such as distillation methods, precipitation/crystallization methods (if applicable), and chromatographic methods (including e.g. flash chromatography and HPLC).

The skilled person in view of his/her common general knowledge is also able to modify a sulfatation procedure applied in the art for the purpose of the present invention, if necessary.

The examples section disclosed herein illustrates an exemplary sulfatation procedure with subsequent neutralization in accordance with step c. of alternative a. of the process of the present invention (cf. Example 3.2).

Alternative b.: Steps b. And c.—Sulfonation-Hydrogenation-Neutralization

In the following, the sulfonation-hydrogenation sequence according to steps b. and c. of alternative b. of the process of the present will be further described.

Step b. of alternative b.: In the process of the present invention according to alternative b., the aryl aliphatic ketone of formula IV: Ar—C(═O)—CH₂—R₃ (IV) obtained from the decarboxylative cross-ketonization step is sulfonated with a sulfonating agent followed by a neutralization with a base XOH or X(OH)₂, thereby obtaining an aryl aliphatic ketone sulfonate of formula VI: Ar—C(═O)—CH(SO₃X)—R₃ (VI) wherein Ar, X and R₃ are as defined above and in the claims.

The sulfonation of aryl aliphatic ketones using a sulfonating agent is known to the skilled person, and e.g. described in WO 2018/229285 A1 (cf. page 43, lines 5-22). Step b. of alternative b. in the process of the present invention can be carried out in accordance with said known procedure as described WO 2018/229285 A1.

In accordance with the known procedure from WO 2018/229285 A1, the sulfonation step in the process of the present invention can be conducted using e.g. gaseous SO₃ as the sulfonating agent, preferably diluted in dry air or dry nitrogen (for example 1 to 5% v/v). In this case, the sulfonation using diluted SO₃ can be performed using a so called falling film reactor. Other sulfonating agents useful in the present invention include chlorosulfonic acid (ClSO₃H), oleum and sulfamic acid (H₃NSO₃). Preferably SO₃ or chlorosulfonic acid (ClSO₃H) is used as the sulfonating agent.

The reaction is typically conducted at temperatures ranging from −20° C. to 200° C., preferably ranging from 0° C. to 120° C. Due to the exothermic nature of the reaction, the sulfonation agent is typically added at lower temperatures (e.g. within the range of −20° C. to 10° C.) to the substrate. After the addition is completed, the temperature is typically increased in order to accelerate the reaction and increase conversion.

The sulfonation can be carried out in solution or neat. The skilled person in view of his/her common general knowledge is able to choose a suitable solvent from the range of standard solvents widely used in organic synthesis, including, but not limited to, e.g. CH₂Cl₂, CHCl₃, CCl₄, pyridine, ethyl acetate, dioxane, diglyme, THF, 2-methyl-THF, hydrocarbons or mixtures thereof.

The skilled person in view of his/her common general knowledge is able to control the progress of the sulfonation reaction using standard techniques, such as NMR.

An ageing step can optionally be included after the sulfonation step in order to achieve full conversion to the sulfonic acid. Such ageing step can e.g. be conducted at a temperature between 15° C. to 200° C.

The sulfonating step is followed by a neutralization step in order to order to complete salt formation of the desired product, i.e. aryl aliphatic ketone sulfonate of formula VI: Ar—C(═O)—CH(SO₃X)—R₃ (VI).

The neutralization can be carried out as already described above in connection with step c. of alternative a. of the process of the present invention.

The skilled person in view of his/her common general knowledge is able to purify the product mixture resulting from the sulfonation and subsequent neutralization step using common purification and separation techniques, such as distillation methods, precipitation/crystallization methods (if applicable), and chromatographic methods (including e.g. flash chromatography and HPLC).

The skilled person in view of his/her common general knowledge is also able to modify a sulfonation procedure applied in the art for the purpose of the present invention, if necessary.

The examples section disclosed herein illustrates an exemplary sulfonation procedure with subsequent neutralization in accordance with step b. of alternative b. of the process of the present invention (cf. Examples 1.1, 2.1) Step c. of alternative b.: The aryl aliphatic ketone sulfonate of formula VI: Ar—C(═O)—CH(SO₃X)—R₃ (VI) wherein Ar, X and R₃ are as defined above and in the claims, is subsequently submitted to a hydrogenation step in the presence of H₂ and a catalyst, thereby obtaining a first compound of formula I, wherein R₁ is OH, R₂ is SO₃X wherein X is as defined above, R₃ is a C₂-C₂₆ aliphatic group; or a second compound of formula I wherein R₁ is H, R₂ is SO₃X wherein X is as defined above and in the claims, R₃ is a C₂-C₂₆ aliphatic group; or a mixture of said first compound and said second compound of formula I.

The catalytic hydrogenation of aryl aliphatic ketones in order to obtain the corresponding aryl aliphatic alcohol is a standard reaction commonly known to the skilled person.

In the context of step c. of alternative b., the catalytic hydrogenation can analogously be carried out as described above in connection with step b. of alternative a. of the process of the present invention.

The skilled person in view of his/her common general knowledge is also able to tune the hydrogenation conditions in favor of obtaining the first compound of formula I and reducing the amount of the second compound of formula I formed or alternatively in favor of the second compound of formula I and reducing the amount of the first compound of formula I. The skilled person in view of his/her common general knowledge is able to control the progress of the reaction using standard techniques, such as NMR.

The skilled person in view of his/her common general knowledge furthermore is able to purify the product mixture resulting from the hydrogenation step using common purification and separation techniques, such as distillation methods, precipitation/crystallization methods (if applicable), and chromatographic methods (including e.g. flash chromatography and HPLC).

The examples section disclosed herein illustrates an exemplary hydrogenation procedure in accordance with step c. of alternative b. of the process of the present invention (cf. Examples 1.2, 2.2).

### Use of the Compounds of Formula I

In view of the advantageous properties of the compounds of the present invention, in terms of their biodegradability profiles as well as their toxicity and environmental profiles, the compounds of the present invention are useful as surfactants. The term “surfactant” as used herein is to be understood in accordance with the common general knowledge of the skilled person.

The present invention shall be illustrated on the basis of the following examples.

Should the disclosure of any patents, patent applications, and publications which are incorporated herein by reference conflict with the description of the present application to the extent that it may render a term unclear, the present description shall take precedence.

### EXAMPLES

### General Remarks

All the reactions are conducted in carefully dried vessels and under an inert argon atmosphere.

Fresh commercial anhydrous CHCl₃ (amylene stabilized) and anhydrous dioxane were used as such.

The phenyl alkyl ketone precursors used in the following examples can be produced from benzoic acid and the corresponding fatty acid following the cross-ketonization procedure described in WO 2018/229285 A1.

The abbreviation “PAK” refers to the phenyl-alkyl ketone precursor obtained from cross-ketonization.

The abbreviation “PAKS” refers to a phenyl-alkyl ketone sulfonate obtained from the phenyl alkyl ketone (“PAK”) precursor by means of sulfonation. The abbreviation “PAS” refers to a phenyl-alcohol sulfonate obtained from the phenyl-alkyl ketone sulfonate (“PAKS”) by means of hydrogenation. The PAS is typically obtained in a product mixture with the corresponding deoxygenated product.

The abbreviation “PAAS” refers to a phenyl-alkyl carbinol sulfate ester obtained from the phenyl-alkyl ketone (“PAK”) precursor by means of a hydrogenation-sulfatation sequence via the phenyl-alkyl secondary alcohol (“PAA”).

### Example 1

1.1 Synthesis of PAKS-C₁₆ from Hexadecanophenone:

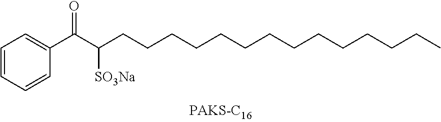

In a 1 L double-jacketed reactor equipped with a mechanical stirrer (propeller with four inclined plows) and baffles, a condenser and a temperature probe are added 38.6 g of 1-phenylhexadecan-1-one (hexadecanophenone) (weight purity>95%, 0.362 mol, 1 eq.) and 257 mL (382 g) of anhydrous CHCl₃. The resulting solution is then heated to 45° C. and stirred at 750 rpm.

In a 500 mL three-neck round-bottom flask equipped with a magnetic stirrer, a condenser and a temperature probe are added 186 mL of anhydrous CHCl₃ (277 g). The solution is cooled down to 0° C., and 93 mL (96 g, 1.09 mol, 3 eq) of 1,4-dioxane is slowly introduced under stirring. Then, 28 mL (49 g, 0.381 mol, 1.05 eq) of chlorosulfonic acid is slowly introduced into the reaction vessel while monitoring reaction media temperature (exothermy). The mixture is then allowed to stir at room temperature during 15 minutes.

The prepared ClSO₃H:dioxane solution in CHCl₃ contained in the 500 mL flask is slowly added over 15 minutes into the 1 L reactor maintained at 45° C. under stirring. The reaction mixture turned yellow upon addition.

Following addition, the temperature of the reaction mixture is then increased to 60° C. and the reaction progress is followed by NMR analysis. After 1 h 30 stirring at 60° C., NMR analysis indicates a ketone conversion around 99 mol % with a crude molar composition of 90 mol % of the desired PAKS-C₁₆, 9 mol % O-sulfatation by-product and 1 mol % of the starting phenyl alkyl ketone. Additional 2.4 ml of chlorosulfonic acid (4.3 g, 0.036 mol, 0.1 eq) is added in order to reach full conversion.

At the end of the reaction (2 h 45 at 60° C. under stirring), the volatiles are distilled off. Following distillation, the temperature of the reaction mass is lowered to 10° C. and 187 mL of water are slowly added into the dark brown residue (exothermy) followed by 200 mL of an aqueous NaOH 10% solution (to reach pH=7).

The solution is then transferred into a 2 L flask and 741 mL of n-butanol is introduced. The biphasic mixture is first decanted and the organic orange phase is evaporated (using n-BuOH azeotropic distillation to help water removal). ¹H NMR analysis in MeOD:D₂O mixture of the crude shows the presence of 3.4 mol % of sodium 2-ethoxyethyl sulfate and 11.3 mol % of sodium ethyl sulfate.

The product is purified by washing the solid several times with CH₃CN (the desired PAKS product being not soluble in CH₃CN). The resulting product is then dried under vacuum to give 140 g of a white solid with a weight composition of 99% of PAKS-C₁₆, 0.5% of O-sulfated by-product (enol sulfate ester), 0.3% of sodium ethyl sulfate and 0.16% of sodium 2-ethoxyethyl sulfate.

The purified yield of PAKS-C₁₆ is 92%.

¹H NMR (MeOD-d₄, 400 MHz) δ (ppm): 8.09 (d, J=7.6 Hz, 2H), 7.65-7.56 (m, 1H), 7.54-7.43 (m, 2H), 4.94 (dd, J=10.6 Hz, J=3.8 Hz, 1H), 2.42-2.03 (m, 2H), 1.49-1.01 (m, 24H), 0.89 (t, J=6.8 Hz, 3H).

¹³C NMR (MeOD-d₄, 101 MHz) δ (ppm): 198.38, 139.6, 134.52, 130.25, 129.74, 66.91, 33.2, 30.9, 30.88, 30.85, 30.79, 30.76, 30.69, 30.68, 30.59, 30.47, 28.57, 23.86, 14.58 (terminal CH₃).

1.2 Synthesis of PAS-C₁₆ from PAKS-C₁₆

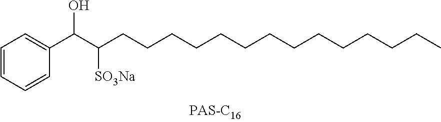

The reaction is conducted in an autoclave under 20 bar hydrogen pressure.

In a 750 ml autoclave reactor equipped with a mechanical stirrer (Rushton turbine) are added a preformed solution obtained by dissolving 70 g (99 wt %, 0.166 mol, 1 eq) of PAKS-C₁₆ in 240 ml (189 g, 36 wt %) ethanol under stirring.

Then 4.24 g of Pd/C 3% (51 wt % moisture content, 3 wt % with respect to the ketone) is added. The reactor is sealed and three nitrogen purges are performed followed by three purges of hydrogen. The reaction mixture is then pressurized under 20 bar of H₂, stirred at 1000 rpm and heated at 90° C. After 4 h 00 reaction time, the reaction mixture is allowed to cool down to room temperature and the reactor is finally depressurized.

NMR analysis in MeOD-d₄ indicates a total conversion of the starting product PAKS-C₁₆ and the following molar composition of the crude: 89 mol % of phenyl alcohol sulfonate, 9 mol % of phenyl alkyl sulfonate (PAKS-C₁₆-HDO) and 2 mol % of sodium ethyl sulfate.

The reactor is drained and rinsed several times with ethanol.

The mixture is filtrated over celite to separate Pd/C catalyst and the filter is rinsed with ethanol and methanol.

The filtrate is evaporated to give 70 g of a white product with the following weight composition: 91 wt % of phenyl alcohol sulfonate, 8 wt % of phenyl alkyl sulfonate and 0.7 wt % of sodium ethyl sulfate corresponding to a yield of 88% for the PAS-C₁₆.

¹H NMR (MeOD-d₄, 400 MHz) δ (ppm): 7.47-7.12 (m, 5H), 5.52 (s, alcohol sulfonate isomer 1, 1H), 4.79 (d, J=9.5 Hz, alcohol sulfonate isomer 2, 1H), 3.45 (dd, J=13.4 Hz, J=3.0 Hz, alkyl sulfonate, 1H), 2.97-2.90 (m, alkyl sulfonate, 1H), 2.89-2.81 (m, alcohol sulfonate, 1H), 2.65 (dd, J=13.7 Hz, J=10.8 Hz, alkyl sulfonate, 1H), 1.95-1.59 (m, 2H), 1.4-0.94 (m, 24H), 0.90 (t, J=6.8 Hz, 3H).

¹³C NMR (MeOD-d₄, 101 MHz) δ (ppm): 143.51, 142.66, 129.45, 129.26, 129.07, 128.13, 126.9, 76.73, 73.03, 67.32, 67.17, 33.21, 30.9, 30.85, 30.79, 30.67, 30.60, 30.36, 30.29, 30.25, 30.03, 29.06, 28.85, 24.24, 23.87, 14.58 (terminal CH₃).

1.3 Synthesis of a Mixture of PAS-C₁₆ and PAS-C₁₆-HDO from PAKS-C₁₆

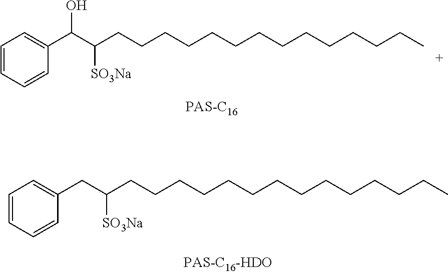

The reaction is conducted in an autoclave under 2 bar hydrogen pressure.

In a 130 ml autoclave reactor equipped with a mechanical stirrer (Rushton turbine) are added a preformed 18 wt % solution obtained by dissolving 8.71 g (99 wt % purity, 0.021 mol) of PAKS-C₁₆ (from example 1.1) in 50 ml (39.58 g) anhydrous ethanol (Sinopharm, H₂O content≤0.2%) under stirring.

Then 1.61 g of Pd/C 5% (J&M, type 5R39, Batch No. C-8750, 45.9 wt % moisture content, 10 wt % dry catalyst with respect to the ketone) is added. The reactor is sealed and three nitrogen purges are performed followed by three purges of hydrogen. The reaction mixture is then pressurized under constant 2 bar of H₂, stirred at 1300 rpm and heated at 80° C. After 7.5 h reaction time, the reaction mixture is allowed to cool down at room temperature and the reactor is finally depressurized.

¹H NMR analysis in MeOD-d₄ indicates a total conversion of the starting material PAKS-C₁₆ and the following molar composition of the crude solid: 22 mol % of phenyl alcohol sulfonate (PAS-C₁₆), 69 mol % of phenyl alkyl sulfonate (PAS-C₁₆HDO) and 9 mol % of the fully hydrogenated by-product 1-cyclohexyl alkyl sulfonate.

The reactor is drained and rinsed several times with anhydrous ethanol.

The mixture is filtrated over celite (Supelco, Celite® 545, particle size 0.02-0.1 mm, Batch No. K53174993 120) to separate Pd/C catalyst and the filter is rinsed with anhydrous ethanol.

The filtrate is evaporated to give 8.47 g of a white product with the following weight composition: 22 wt % of phenyl alcohol sulfonate (PAS-C₁₆), 68 wt % of phenyl alkyl sulfonate (PAS-C₁₆-HDO) and 9 wt % of the fully hydrogenated by-product 1-cyclohexyl alkyl sulfonate.

¹H NMR (300 MHz, MeOD-d₄,): δ (ppm) 7.42-7.15 (m, 5H), 5.52 (s, alcohol, sulfonate isomer 1, 1H), 4.79 (d, J=9.5 Hz, alcohol sulfonate isomer 2, 1H), 3.45 (dd, J=13.6 Hz, J=3.1 Hz, alkyl sulfonate, 1H), 2.96-2.82 (m, alkyl sulfonate and alcohol sulfonate, 2H), 2.64 (dd, J=13.5 Hz, J=10.8 Hz, alkyl sulfonate, 1H), 1.94-0.94 (m, 26H), 0.90 (t, J=7.0 Hz, 3H).

¹³C{¹H} NMR (75 MHz, MeOD-d₄): δ (ppm) 141.1, 130.2, 129.36, 129.28, 129.1, 128.9, 128.0, 127.2, 126.7, 76.5, 72.8, 67.0, 63.0, 38.1, 33.0, 30.8, 30.74, 30.72, 30.67, 30.63, 30.57, 30.52, 30.4, 30.3, 28.3, 24.0, 23.7, 14.5 (terminal CH₃).

### Example 2

2.1 Synthesis of PAKS-C₁₈ from Octadecanophenone

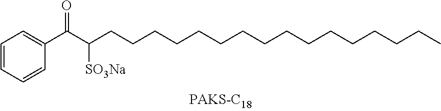

In a 500 mL double-jacketed reactor equipped with a mechanical stirrer (propeller with four inclined plows) and baffles, a condenser and a temperature probe are added 50 g of 1-phenyloctadecan-1-one (octadecanophenone) (0.145 mol, 1 eq.) and 168 mL (250 g) of anhydrous CHCl₃. The solution is then heated to 40° C. and stirred at 400 rpm.

In a 500 mL three-neck round-bottom flask equipped with a magnetic stirrer and a temperature probe are added 74.5 mL of anhydrous CHCl₃ (111 g). The solution is cooled down to 0° C., and 37.3 mL (38.4 g, 0.435 mol, 3 eq) of 1,4-dioxane is slowly introduced under stirring (350 rpm). Then, 10.1 mL (17.75 g, 0.152 mol, 1.05 eq) of chlorosulfonic acid (ClSO₃H) is slowly introduced into the reaction vessel over 10 minutes while monitoring reaction media temperature (exothermy). The mixture is then allowed to stir at room temperature during 30 minutes.

The prepared ClSO₃H:dioxane solution in CHCl₃ contained in the 500 mL flask is slowly added in 35 minutes into the 500 mL reactor maintained at 40° C. under stirring. The reaction mixture turned yellow upon addition. Following addition, the temperature of the reaction mixture is then increased to 60° C. and the reaction progress is followed by NMR analysis. After 1 h 30 stirring at 60° C., NMR analysis indicates a complete conversion of ketone.

The volatiles are then removed by distillation (60° C., 20 mbar) and 60 mL of water is carefully added at room temperature to the obtained residue (exothermic). Finally, 64 mL of an aqueous 10% NaOH solution is added to the mixture under stirring at room temperature in order to reach pH=7.

The obtained solution is then transferred into a 1 L flask and water is removed under vacuum using n-BuOH azeotropic distillation to help water removal.

After evaporation, 66.5 g of a white solid is obtained with a weight composition of 93 wt % of PAKS-C₁₈, 6.2 wt % of O-sulfated by-product (enol sulfate ester), and 1.1 wt % of residual starting ketone. The yield of PAKS-C₁₈ is quantitative without taking into account the presence of salts.

¹H NMR (MeOD-d₄, 400 MHz) δ (ppm): 8.09 (d, J=7.7 Hz, 2H), 7.64-7.56 (m, 1H), 7.54-7.46 (m, 2H), 7.42-7.26 (m, enol sulfate ester, 4H), 6.22 (t, J=7.1 Hz, enol sulfate ester, 1H), 4.93 (dd, J=10.7 Hz, J=3.5 Hz, 1H), 2.43-2.36 (m, enol sulfate ester, 2H), 2.38-2.26 (m, 2H), 2.18-2.04 (m, 2H), 1.49-1.00 (m, 28H), 0.89 (t, J=6.7 Hz, 3H).

¹³C NMR (MeOD-d₄, 101 MHz) δ (ppm): 197.91, 139.73, 134.39, 130.25, 129.70, 67.04, 33.22, 30.9, 30.87, 30.77, 30.70, 30.67, 30.61, 30.49, 28.59, 23.88, 14.58 (terminal CH₃).

2.2 Synthesis of PAS-C₁₈ from PAKS-C₁₈

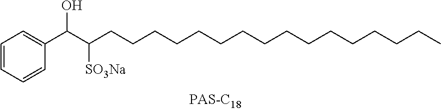

The reaction is conducted in an autoclave under 20 bar of hydrogen pressure.

In a 100 ml autoclave reactor equipped with a mechanical stirrer (Rushton turbine) are added 18 g (0.040 mol, 1 eq) of PAKS-C₁₈ and 1.1 g of Pd/C 3% (51 wt % moisture content, 3 wt % with respect to the ketone). Then 70 mL (55.3 g, 33 wt %) of ethanol is added to the solids.

The reactor is sealed and three nitrogen purges are performed followed by three purges of hydrogen. The reaction mixture is then pressurized under 20 bar of H₂, stirred at 1000 rpm and heated at 90° C. After 5 h 00 reaction time, the reaction mixture is allowed to cool down to room temperature and the reactor is finally depressurized.

The reactor is drained and rinsed several times with ethanol and CHCl₃. The mixture is filtrated over paper filter to separate Pd/C catalyst and the filter is rinsed with ethanol. The filtrate is evaporated to give 16.10 g of a white product after thorough drying.

The weight composition of the product is the following: 83 wt % of phenyl alcohol sulfonate, 10 wt % of phenyl alkyl sulfonate, 7 wt % of octadecylbenzene, and 0.5 wt % of sodium ethyl sulfate.

The yield taking into account the purity is 83%.

¹H NMR (MeOD-d₄, 400 MHz) δ (ppm): 7.45-7.10 (m, 5H), 5.52 (s, alcohol sulfonate isomer 1, 1H), 4.79 (d, J=9.5 Hz, alcohol sulfonate isomer 2, 1H), 3.45 (dd, J=14.2 Hz, J=3.1 Hz, alkyl sulfonate, 1H), 2.96-2.90 (m, alkyl sulfonate, 1H), 2.89-2.81 (m, alcohol sulfonate, 1H), 2.65 (dd, J=13.5 Hz, J=10.7 Hz, alkyl sulfonate, 1H), 1.85-1.50 (m, 2H), 1.4-0.8 (m, 28H), 0.90 (t, J=6.8 Hz, 3H).

### Example 3

3.1 Synthesis of PAA-C₁₆ from PAK-C₁₆

The phenyl-alkyl alcohol compound PAA-C₁₆ can be obtained from the corresponding phenyl-alkyl ketone (PAK-C₁₆) precursor through catalytic hydrogenation in accordance with the procedure described in US 2020/0339748 A1 (cf. e.g. paragraphs [0265]-[0270] of US 2020/0339748 A1).

3.2 Synthesis of PAAS-C₁₆ from PAA-C₁₆

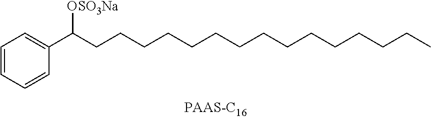

In a 100 mL three necked round bottom flask equipped with a condenser, a temperature probe, a mechanical stirring device (propeller with 4 inclined plows), baffles and a heating plate are added 100 mL of pyridine (98 g, 1.24 mole, 8 eqs).

The reaction mass is then cooled down to 0° C. and 11.6 mL of ClSO₃H (20.33 g, 0.175 mole, 1.1 eqs) is progressively added under stirring at 400 rpm (strong exothermy).

The mixture is then heated to 50° C. and 50 g of melted phenylhexadecan-1-ol (0.157 mole, 1 eq.) is progressively added into the reaction mixture thanks to an insulated dropping funnel maintained at 90° C. under stirring at 500 rpm during 20 minutes.

At the end of the addition, the reaction mixture is heated to 60° C. and the reaction progress is followed by ¹H NMR analysis. After 2 h 00 stirring at 60° C., ¹H NMR analysis shows a complete and selective conversion of the starting alcohol to the desired sulfuric ester.

The volatiles are then removed through distillation at 60° C. under vacuum (10 mbar) and 180 mL of water is added to the white residue. The obtained dispersion is then stirred at room temperature and 2.2 equivalents (with respect to HSO₃Cl) of a NaOH aqueous solution (5M) is progressively added to the mixture for neutralization in order to reach pH=6.

800 mL of CH₃CN is then added to the aqueous mixture in order to precipitate out the product. The white precipitate is filtered and is washed several times with toluene followed by CHCl₃ in order to remove pyridinium salts to undetectable levels by NMR.

At this stage, the product (62.3 g) contains some residual amounts of inorganic salts. The white solid is dissolved in 200 mL of EtOH in order to precipitate the inorganic salts which are filtered out. The solid is washed several times with 50 mL of EtOH to extract the product. Evaporation of solvent affords a first crop of product. The ethanol filtrate is concentrated and CH₃CN is added into the concentrated solution in order to collect additional product. The obtained solid is washed several times with CH₃CN and CHCl₃.

The two crops are then gathered and dried under vacuum to remove traces of solvent and to afford 40.27 g of white product corresponding to 67% isolated yield.

¹H NMR (MeOD-d₄, 400 MHz) δ (ppm): 7.45-7.15 (m, 5H), 5.26 (t, J=6.7 Hz, 1H), 2.04-1.88 (m, 1H), 1.88-1.72 (m, 1H), 1.55-1.05 (m, 26H), 0.90 (t, J=6.9 Hz, 3H).

¹³C NMR (MeOD-d₄, 101 MHz) δ (ppm): 143.23, 129.22, 128.53, 127.75, 82.13, 38.78, 33.23, 30.91, 30.88, 30.80, 30.76, 30.65, 30.63, 26.38, 23.89, 14.59 (terminal CH₃).

### Example 4

4.1 Synthesis of PAKS-C₁₂₋₁₈ from PAK-C₁₂₋₁₈

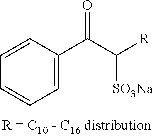

All the reactions are conducted in carefully dried vessels and under an inert argon atmosphere.

Fresh commercial anhydrous CHCl₃ and 1,4-dioxane were used as such.

In a 1 L double-jacketed reactor equipped with a mechanical stirrer (propeller with four inclined plows) and baffles, a condenser and a temperature probe are added:
50 g of phenyl alkyl ketone C₁₂₋₁₈ cut (0.18 mole, 1 eq. with the following side alkyl chain length distribution: C₁₀=0.8 wt %, C₁₂=57.3 wt %, C₁₄=21.1 wt %, C₁₆=9.8 wt % and C₁₈=10.9 wt %)
112 mL (167 g, 23 wt % reactant concentration in solution) of anhydrous CHCl₃

The mixture is heated to 45° C. and stirred at 600 rpm until complete solubilization of the solid at room temperature.

In a 500 mL three-neck round-bottom flask equipped with a magnetic stirrer, a condenser and a temperature probe, 92 mL of anhydrous CHCl₃ (137.5 g) are introduced.

The solution is allowed to cool down at 0° C., stirred at 600 rpm and 1,4-dioxane is introduced into the vessel (46 mL, 47.5 g, 0.54 mole, 3 eq. with respect to ketone) (slightly exothermic).

Chlorosulfonic acid (15 mL, 26.5 g, 0.225 mole, 1.05 eq. with respect to the ketone) is then progressively added into the solution thanks to an insulated dropping funnel during 10 minutes while controlling the addition rate to prevent the reaction mass temperature to increase over 10° C. (exothermic). At the end of the addition, the reaction mixture is stirred during 15 minutes at 0° C. and allowed to warm-up at room temperature.

The obtained HSO₃Cl:dioxane chloroform solution is slowly added in 15 min through a teflon cannula into the 1 L reactor which is maintained at 45° C. Upon addition the reaction mixture turns yellow readily.

At the end of the addition, the temperature of the reactor is increased at 60° C. and after 2 h 30 stirring at 600 rpm, ¹H NMR analysis (in MeOD) indicated a ketone conversion level around 94%.

In order to reach full conversion 2.4 mL of chlorosulfonic acid (4.23 g, 0.036 mol, 0.2 eq.) is finally added. The reaction is allowed to stir at 60° C. until conversion level is over 99% which requires 3 additional hours of stirring. At this stage the molar composition of the solid is 94 mol % ketone sulfonic acid, 2.5 mol % enol sulfuric acid ester and only 0.2 mol % of the starting phenyl alkyl ketone.

The 1,4-dioxane and the chloroform are then distilled off under vacuum using 3 traps (1 empty trap maintained at −78° C. and 2 traps filled with 10 wt % NaOH solution) and with the collecting flask from the distillation apparatus cooled down at 0° C. The crude mixture is maintained at 60° C. and the vacuum is progressively decreased by controlling foam formation. Upon distillation, the mixture becomes very viscous; therefor stirring rate is decreased down to 400 rpm.

At the end of the distillation, atmospheric pressure is reestablished and the temperature in the pot is lowered to 10° C. and 120 ml of water are carefully added to the dark brown residue (strong exothermy: internal temperature=35° C. at the end of addition). pH of the resulting orange solution is adjusted to ˜7 by addition of 90 ml NaOH 10%.

The solution is then transferred into a 2 L flask and 366 mL of n-butanol are introduced. The phases are separated and the organic orange phase is evaporated along with water thanks to azeotropic distillation.

The final product is purified by dissolving the solid in 200 mL of ethanol (to precipitate salts by-products) followed by a filtration and solvent evaporation.

At this stage ¹H NMR analysis shows that the product still contains 2.4 mol % sodium 2-ethoxyethyl sulfate and 10 mol % sodium ethyl sulfate, therefor the product is solubilized in 500 ml diethyl ether and washed with 300 mL of a saturated NaCl aqueous solution. The organic phase is separated and the aqueous phase is re-extracted with 500 mL diethyl ether.

The two organic phases are combined, dried over Na₂SO₄, filtered and evaporated at 65° C. under vacuum (10 mbar). At this stage, the product still contains NaCl traces which are definitively removed by precipitation using 300 mL of ethanol.

Solvent evaporation after filtration results to 61 g of white solid with the following weight composition: 95.6% of PAKS C₁₂₋₁₈, 4% of enol-sulfate ester, 0.1% of sodium ethyl sulfate and 0.3% of sodium ethoxyethyl sulfate.

The purified yield of PAKS C₁₂₋₁₈ is 89%.

¹H NMR (CDCl₃, 400 MHz) δ (ppm): 8.11-8.08 (d, 2H), 7.63-7.58 (m, 1H), 7.53-7.48 (m, 2H), 4.96-4.2 (dd, 1H), 2.35-2.09 (m, 2H), 1.34-1.19 (m, 19H), 0.89 (t, J=6.8 Hz, 3H).

¹³C NMR (CDCl₃, 101 MHz) δ (ppm): 198.27, 139.61, 134.51, 130.25, 129.73, 66.91, 33.2, 30.89, 30.86, 30.77, 30.73, 30.7, 30.54, 30.48, 28.57, 23.86, 14.58 (terminal CH₃).

4.2 Synthesis of a Mixture of PAS-C₁₂₋₁₈ and PAS-C₁₂₋₁₈-HDO from PAKS-C₁₂₋₁₈

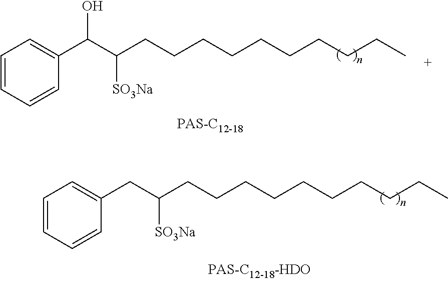

The reaction is conducted in an autoclave under 2 bar hydrogen pressure.

In a 130 ml autoclave reactor equipped with a mechanical stirrer (Rushton turbine) are added a preformed 18 wt % solution obtained by dissolving 12.19 g (99 wt % purity) of PAKS-C₁₂₋₁₈ in 70 ml (55.4 g) anhydrous ethanol (Sinopharm, H₂O content≤0.2%) under stirring.

Then 3.84 g of Pd/C 5% (J&M, type 5SA390000, Batch No. S321F02541, 52.4 wt % moisture content, 15 wt % dry catalyst with respect to the ketone) is added. The reactor is sealed and three nitrogen purges are performed followed by three purges of hydrogen. The reaction mixture is then pressurized under constant 2 bar of H₂, stirred at 1300 rpm and heated at 80° C. After 7.5 h reaction time, the reaction mixture is allowed to cool down to room temperature and the reactor is finally depressurized.

¹H NMR analysis in MeOD-d₄ indicates a total conversion of the starting product PAKS-C₁₂₋₁₈ and the following molar composition of the crude solid: 16 mol % of phenyl alcohol sulfonate (PAS-C₁₂₋₁₈), 73 mol % of phenyl alkyl sulfonate (PAS-C₁₂₋₁₈-HDO) and 11 mol % of the fully hydrogenated 1-cyclohexyl alkyl sulfonate by-product.

The reactor is drained and rinsed several times with anhydrous ethanol.

The mixture is filtrated over celite (Supelco, Celite® 545, particle size 0.02-0.1 mm, Batch No. K53174993 120) to separate Pd/C catalyst and the filter is rinsed with anhydrous ethanol.

The filtrate is evaporated to give 11.5 g of a white product with the following weight composition: 17 wt % of phenyl alcohol sulfonate (PAS-C₁₂₋₁₈), 72 wt % of phenyl alkyl sulfonate (PAS-C₁₂₋₁₈-HDO and 11 wt % of the fully hydrogenated 1-cyclohexyl alkyl sulfonate by-product.

¹H NMR (300 MHz, MeOD-d₄,): δ (ppm) 7.43-7.12 (m, 5H), 5.52 (s, alcohol, sulfonate isomer 1, 1H), 4.79 (d, J=9.5 Hz, alcohol sulfonate isomer 2, 1H), 3.45 (dd, J=13.7 Hz, J=3.1 Hz, alkyl sulfonate, 1H), 2.97-2.83 (m, alkyl sulfonate and alcohol sulfonate, 2H), 2.65 (dd, J=13.6 Hz, J=10.7 Hz, alkyl sulfonate, 1H), 2.57 (t, J=8.1 Hz, alkylbenzene, 1H), 1.89-0.87 (m, 26H).

4.3 Synthesis of PAA-C₁₂₋₁₈ from PAK-C₁₂₋₁₈

The phenyl-alkyl alcohol compound PAA-C₁₂₋₁₈ can be obtained from the corresponding phenyl-alkyl ketone cut (PAK-C₁₂₋₁₈) precursor through catalytic hydrogenation in accordance with the procedure described in US 2020/0339748 A1 (cf. e.g. paragraphs [0265]-[0270] of US 2020/0339748 A1).

4.4 Synthesis of PAAS-C₁₂₋₁₈ from PAA-C₁₂₋₁₈

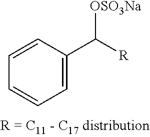

The reaction is conducted in carefully dried vessels and under an inert argon atmosphere.

Fresh commercial anhydrous pyridine was used as such.

In a 1 l double-jacketed reactor equipped with a mechanical stirrer (propeller with four inclined plows) and baffles, a condenser and a temperature probe are added 175 ml (171.15 g, 2.16 mol, 4 eq) of pyridine.

The reaction mass is then cooled down to 0° C. and 39.9 ml (69.8 g, 0.593 mol, 1.1 eq) of chlorosulfonic acid is progressively added under stirring at 300 rpm while keeping the temperature below 40° C. (strong exothermy).

The mixture is then heated to 50° C. and 151 g of melted phenyl alkyl carbinol C₁₂₋₁₈ cut (from example 4.3) (0.539 mole, 1 eq. with the following side alkyl chain length distribution: C₁₀: 0.8 wt % C₁₂: 57.3 wt %, C₁₄: 21.1 wt %, C₁₆: 9.8 wt % and C₁₈: 10.9 wt %) is progressively added into the reaction mixture under stirring at 500 rpm during 10 minutes thanks to an insulated dropping funnel maintained at 30° C.

At the end of the addition, the reaction mixture is heated to 60° C. and the reaction progress is followed by ¹H NMR analysis. After 1 h 00 stirring at 60° C., ¹H NMR analysis shows a complete and selective conversion of the starting alcohol to the desired sulfate ester.

The volatiles are then removed through distillation at 70° C. under vacuum (10 mbar) during 1 h 45. The residue containing the sulfate ester (with pyridinium as counter-cation) is dissolved in 1.5 L Et₂O followed by the addition of 1.5 L aqueous NaOH solution (0.5M). The yellow aqueous phase containing the product is separated from the organic phase containing organic impurities.

1 L of diethyl ether is then added to the aqueous phase followed by the addition of sodium chloride until saturation of the aqueous phase. Upon addition of sodium chloride, ion exchange occurs and the desired sodium sulfate ester migrates to the organic phase which is separated, dried over MgSO₄ and filtered. The MgSO₄ solid is rinsed several times with MeOH.

At this stage, the sulfate ester product still contains traces of pyridinium counter-ion, therefor NaHCO₃ is introduced into the solution. The mixture is stirred at room temperature during 30 minutes, the solid is filtered and the filtrate is evaporated at 40° C. under vacuum (10 mbar) until a wax forms.

In order to remove residual traces of solvent from the solid while avoiding product degradation, a solution of 100 g MTBE containing 3 wt % of diisopropylamine is added to the solid. The obtained solution is warmed at 40° C. and vacuum is gradually applied until the product solidifies in the form of a white solid. The product is dried under vacuum at 40° C. (10 mbar) for 1 hour to afford 208 g of white product with 96 mol % purity corresponding to 96% isolated yield.

¹H NMR (MeOD-d₄, 400 MHz) δ (ppm): 7.45-7.15 (m, 5H), 5.26 (t, J=6.7 Hz, 1H), 2.04-1.88 (m, 1H), 1.88-1.72 (m, 1H), 1.55-1.05 (m, 21H), 0.90 (t, J=6.9 Hz, 3H).

¹³C NMR (MeOD-d₄, 101 MHz) δ (ppm): 143.23, 129.22, 128.53, 127.75, 82.13, 38.78, 33.23, 30.91, 30.88, 30.80, 30.76, 30.65, 30.63, 26.38, 23.89, 14.59 (terminal CH3).

### Example 5

### 5.1 Biodegradability Assessment Using the 301 F OECD Protocol (Standard Protocol)

A measured volume of inoculated mineral medium, containing a known concentration of test substance in order to reach about 50 to 100 mg ThOD/l (ThOS=Theoretical Oxygen Demand) as the nominal sole source of organic carbon is stirred in a closed culture BOD (biological oxygen demand) flask (Oxitop™respirometric flask) at a constant temperature (20±2° C.) for up to 28 days in order to assess the biodegradability of the test sample.

Evolved carbon dioxide is absorbed by pellets of sodium or potassium hydroxide present in the head space of the flask. The amount of oxygen taken up by the microbial population (=oxygen consumption expressed in mg/l) during the biodegradation process (biological oxidation of the test substance) decreases the pressure of the head space (ΔP measured by the pressure switch) and is mathematically converted in mg O₂ consumed/litre.

The inoculum corresponds to a municipal activated sludge washed in mineral medium (ZW media) in order to decrease the Dissolved Oxygen Carbon (DOC) content.

Two types of control solutions are used for validation purpose: one containing the reference substance sodium acetate only and a second one containing the reference substance and the test substance as a toxicity control. The reference substance is tested in a flask at a nominal concentration of 129 mg/l corresponding to 100 mg ThOD/l in order to check the viability of the inoculum.

### 5.2 Biodegradability Assessment Using the Emulsion Protocol

As the compounds and mixtures of compounds of the present invention are usually poorly soluble in water, a specific protocol named the “emulsion protocol” has been developed by the inventors for the biodegradability assessment. This protocol enables increasing the solubility of the poorly water-soluble substances in the aqueous phase comprising the inoculum and thus its bioavailability. In the emulsion protocol, the test substance is added into the BOD flask containing the inoculated mineral medium through a stock solution made in an emulsion.

The emulsion used in the emulsion protocol is a 50/50 v/v mixture of a stock solution of the test substance dissolved in a non-biodegradable surfactant (Synperonic® PE 105 at 1 g/l), which is mixed with a mineral silicone oil AR 20 (Sigma). In order to achieve dissolution of the test substance in the non-biodegradable surfactant solution, magnetic stirrer agitation followed by ultrasonication may be applied. Once dissolution is completed, the non-biodegradable surfactant solution comprising the test substance is mixed with the mineral silicone oil at a 50/50 volume/volume ratio. This emulsion is maintained by magnetic stirrer agitation and added to the BOD flask containing the inoculated mineral medium until the required test substance concentration has been reached.

### 5.3 Biodegradability Results

The compounds of the present invention have been evaluated accordingly either using the standard protocol (without emulsion) or using the emulsion protocol, the results are shown in the following Table 1 below:

TABLE 1

| | | | | % biodeg | % biodeg |
|---|---|---|---|---|---|
| | | | | (28 d) | (28 d) |
| | Test | Lag | | (Standard | (Emulsion |
| Example | Substance | phase | | protocol) | protocol) |
| 1.1 (Comp) | PAKS-C₁₆ | 10 | days | 49% | — |
| 1.2 | PAS-C₁₆ | 4 | days | 45% | — |
| 1.3 | PAS-C₁₆ + PAS- | 3 | days | 44% | 48% |
| | C₁₆-HDO | | | | |
| 2.1 (Comp) | PAKS-C₁₈ | 8 | days | 52% | — |
| 2.2 | PAS-C₁₈ | 3 | days | 57% | — |
| 3.2 | PAAS-C₁₆ | 3 | days | 55% | 66% |
| 4.2 | PAS-C₁₂₋₁₈ + | 5 | days | 54% | 33% |
| | PAS-C₁₂₋₁₈-HDO | | | | |
| 4.4 | PAAS-C₁₂₋₁₈ | 4 | days | 69% | 64% |

The compounds of the present invention display a good biodegradability profile. It has also been observed that PAS compounds in accordance with the present invention compared to PAKS compounds (“Comp”=comparative=not in accordance with the present invention) display a better biodegradability profile (as indicated by a shorter lag phase and higher biodegradation rate after 28 days; the lag phase is the period when the bacteria are adapting themselves to the test substance, i.e. the period that precedes the beginning of the mineralization of the test substance).

### 5.4 Surface Activity of the Compounds of the Present Invention.

Critical micelle concentrations (CMC) and surface tensions (ST) were measured for aqueous solutions of the surfactants of the present invention at room temperature using a force tensiometer (Sigma 700/701). Plots of surface tension against surfactant concentration were drawn allowing to determine CMC and surface tension at the CMC. Using the Gibbs adsorption equation it is possible to determine the surface excess as well as the area occupied by the surfactant molecule at the surface (as a reference see for example “Industrial Utilization of Surfactants: principles and practice”, M. J. Rosen and M. Dahanayake, 2000, ISBN 1893997111) which are important parameters to characterize a surfactant. Results are summarized in the table below:

| | | Surface | | Cross- |
|---|---|---|---|---|
| | | tension | Surface | sectional |
| | CMC | (at CMC) | excess | area |
| Surfactant | (mM) | mN/m | (mmol/m²) | (nm²) |
| PAKS-C₁₆ | 0.23 | 44 | 0.0026 | 0.64 |
| Example 1.1 (Comp) | | | | |
| PAS-C₁₆ | 0.22 | 36 | 0.0031 | 0.53 |
| Example 1.2 | | | | |

Those results show that PAKS-C₁₆ and PAS-C₁₆ possess similar CMC values (˜0.2 mM) but the latter displays a tighter packing at the interface characterized by a lower cross-sectional area (0.53 nm² vs. 0.64), a higher surface excess and therefor an improved surface activity as confirmed by the lower surface tension displayed at the CMC for PAS-C₁₆ (36 mN/m vs. 44).

1. Compound of formula I 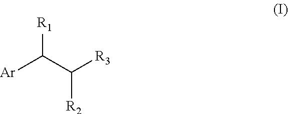
wherein Ar represents an aryl group;
R₁ is H or a group of formula OR wherein R is H or SO₃X wherein X is an alkali metal, alkali-earth metal or ammonium of general formula R′R″R″′R″″N wherein R′, R″, R″′ and R″″ are independently chosen from hydrogen or a hydrocarbyl group which can be optionally substituted and/or interrupted by one or more heteroatom containing groups;
R₂ is H or SO₃X wherein X is an alkali metal, alkali-earth metal or ammonium of general formula R′R″R″′R″″N wherein R′, R″, R′″ and R″″ are independently chosen from hydrogen or a hydrocarbyl group which can be optionally substituted and/or interrupted by one or more heteroatom containing groups;
R₁ and R₂ are different groups;
when R₂ is SO₃X, R₁ is H or OR with R is H;
when R₂ is H, R₁ is OR with R is SO₃X; and
R₃ is a C₂-C₂₆ aliphatic group.

2. The compound according to claim 1, wherein R₃ is a C₂-C₁₈ aliphatic group.

3. The compound according to claim 1, wherein R₃ is an alkyl group.

4. The compound according to claim 1, wherein Ar is a phenyl group.

5. The compound according to claim 1, wherein X is sodium.

6. The compound according to claim 1 wherein:
R₁ is OSO₃X;
R₂ is H; and
R₃ is a linear alkyl group C₆ to C₁₆.

7. The compound according to claim 1, wherein:
R₁ is OH;
R₂ is SO₃X; and
R₃ is a linear alkyl group C₆ to C₁₆.

8. The compound according to claim 1, wherein:
R₁ is H;
R₂ is SO₃X; and
R₃ is a linear alkyl group C₆ to C₁₆.

9. A composition comprising a mixture of two or more compounds of formula I as defined in claim 1.

10. The composition according to claim 9 comprising a mixture of a first compound of formula I wherein:
R₁ is OH;
R₂ is SO₃X; and
R₃ is a linear alkyl group C₆ to C₁₆;
and a second compound of formula I wherein:
R₁ is H;
R₂ is SO₃X; and
R₃ is a linear alkyl group C₆ to C₁₆.

11. A composition comprising at least one compound of formula I as defined in claim 1 and at least one further surfactant, wherein the at least one further surfactant is not a compound of formula I.

12. A process for the preparation of a compound of formula I as defined in claim 1, wherein said process starts from a mixture of Ar—COOH (II) and R₃—CH₂—COOH (III) to produce an aryl aliphatic ketone of formula IV: Ar—C(═O)—CH₂—R₃ (IV), and further comprises the derivatization of the aryl aliphatic ketone of formula IV: Ar—C(═O)—CH₂—R₃ (IV) by a hydrogenation-sulfatation sequence or a sulfonation-hydrogenation sequence to produce the compound of formula I.

13. The process according to claim 12, wherein said process comprises the following steps:
a. Decarboxylative cross-ketonization between Ar—COOH (II) and R₃—CH₂—COOH (III), in presence of a metal based catalyst with the formation of an aryl aliphatic ketone of formula IV: Ar—C(═O)—CH₂—R₃ (IV);
b. Hydrogenation of the aryl aliphatic ketone of formula IV obtained at step a. in presence of H₂ and a catalyst with the formation of a secondary alcohol of formula V: Ar—CH(OH)—CH₂—R₃ (V); and
c. Sulfatation of the secondary alcohol of formula V obtained at step b. with a sulfating agent followed by a neutralization with XOH or X(OH)₂ with the formation of the compound of formula I wherein:
R₁ is OSO₃X;
R₂ is H; and
R₃ is a C₂-C₂₆ aliphatic group.

14. The process according to claim 5, wherein said process comprises the following steps:
a. Decarboxylative cross-ketonization between Ar—COOH (II) and R₃—CH₂—COOH (III), in presence of a metal based catalyst with the formation of an aryl aliphatic ketone of formula IV: Ar—C(═O)—CH₂—R₃ (IV);
b. Sulfonation of the aryl aliphatic ketone of formula IV obtained at step a. with a sulfonating agent followed by a neutralization with XOH or X(OH)₂ with the formation of an aryl aliphatic ketone sulfonate of formula VI: Ar—C(═O)—CH(SO₃X)—R₃ (VI); and
c. Hydrogenation of the aryl aliphatic ketone sulfonate of formula VI obtained at step b. in presence of H₂ and a catalyst with the formation of a first compound of formula I wherein:
R₁ is OH;
R₂ is SO₃X; and
R₃ is a C₂-C₂₆ aliphatic group;
or a second compound of formula I wherein:
R₁ is H;
R₂ is SO₃X; and
R₃ is a C₂-C₂₆ aliphatic group;
or a mixture of said first compound and said second compound of formula I.

15. The process according to claim 13, wherein the sulfating agent is selected from the group consisting of SO₃, chlorosulfonic acid (ClSO₃H), oleum and sulfamic acid (H₃NSO₃).

16. The process according to claim 14, wherein the sulfonating agent is selected from the group consisting of SO₃, chlorosulfonic acid (ClSO₃H), oleum and sulfamic acid (H₃NSO₃).

17. The compound of formula I according to claim 1, wherein the compound is a surfactant.

18. The compound according to claim 1, wherein R₃ is a C₆-C₁₆ aliphatic group.

19. The compound according to claim 1, wherein R₃ is a linear alkyl group.

20. The process according to claim 16, wherein the sulfonating agent is selected from the group consisting of SO₃ and chlorosulfonic acid (ClSO₃H).
